(19) United States
(12) Patent Application Publication
(10) Pub. No.: US 2008/0287487 A1
(43) Pub. Date: Nov. 20, 2008

(54) Muscarinic Acetylcholine Receptor Antagonists

(76) Inventors: Anthony William James Cooper, Hertfordshire (GB); Dramane I. Laine, King of Prussia, PA (US); Michael R. Palovich, King of Prussia, PA (US); Sonia M. Thomas, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/570,981

(22) PCT Filed: Jun. 30, 2005
(86) PCT No.: PCT/US05/23743

§ 371 Date: Dec. 20, 2006

Related U.S. Application Data
(60) Provisional application No. 60/584,011, filed on Jun. 30, 2004.

(51) Int. Cl. A61K 31/44; C07D 453/02; A61P 11/00

(52) U.S. Cl. 514/305; 546/133

(57) ABSTRACT

Muscarinic Acetylcholine Receptor Antagonists and methods of using them are provided.

### FIELD OF THE INVENTION

This invention relates to bi-aryl 8-azoniabicyclo[3.2.1]octane compounds, pharmaceutical compositions, and use thereof in treating muscarinic acetylcholine receptor mediated diseases of the respiratory tract.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs where they mediate many of the vital functions. Muscarinic receptors can mediate both inhibitory and excitatory actions. For example, in smooth muscle found in the airways, M₃ mAChRs mediate contractile responses. For review, please see Caulfield (1993 Pharmac. Ther. 58:319-79).

In the lungs, mAChRs have been localized to smooth muscle in the trachea and bronchi, the submucosal glands, and the parasympathetic ganglia. Muscarinic receptor density is greatest in parasympathetic ganglia and then decreases in density from the submucosal glands to tracheal and then bronchial smooth muscle. Muscarinic receptors are nearly absent from the alveoli. For review of mAChR expression and function in the lungs, please see Fryer and Jacoby (1998 Am J Respir Crit Care Med 158(5, pt 3) S 154-60).

Three subtypes of mAChRs have been identified as important in the lungs, M₁, M₂ and M₃ mAChRs. The M₃ mAChRs, located on airway smooth muscle, mediate muscle contraction. Stimulation of M₃ mAChRs activates the enzyme phospholipase C via binding of the stimulatory G protein Gq/11 (Gs), leading to liberation of phosphatidyl inositol-4,5-bisphosphate, resulting in phosphorylation of contractile proteins. M₃ mAChRs are also found on pulmonary submucosal glands. Stimulation of this population of M₃ mAChRs results in mucus secretion.

M₂ mAChRs make up approximately 50-80% of the cholinergic receptor population on airway smooth muscles. Although the precise function is still unknown, they inhibit catecholaminergic relaxation of airway smooth muscle via inhibition of cAMP generation. Neuronal M₂ mAChRs are located on postganglionlic parasympathetic nerves. Under normal physiologic conditions, neuronal M₂ mAChRs provide tight control of acetylcholine release from parasympathetic nerves. Inhibitory M₂ mAChRs have also been demonstrated on sympathetic nerves in the lungs of some species. These receptors inhibit release of noradrenaline, thus decreasing sympathetic input to the lungs.

M₁ mAChRs are found in the pulmonary parasympathetic ganglia where they function to enhance neurotransmission. These receptors have also been localized to the peripheral lung parenchyma, however their function in the parenchyma is unknown.

Muscarinic acetylcholine receptor dysfunction in the lungs has been noted in a variety of different pathophysiological states. In particular, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation (Fryer et al. 1999 Life Sci 64 (6-7) 449-55). This mAChR dysfunction results in airway hyperreactivity and hyperresponsiveness mediated by increased stimulation of M₃ mAChRs. Thus the identification of potent mAChR antagonists would be useful as therapeutics in these mAChR-mediated disease states.

COPD is an imprecise term that encompasses a variety of progressive health problems including chronic bronchitis, chronic bronchiolitis and emphysema, and it is a major cause of mortality and morbidity in the world. Smoking is the major risk factor for the development of COPD; nearly 50 million people in the U.S. alone smoke cigarettes, and an estimated 3,000 people take up the habit daily. As a result, COPD is expected to rank among the top five as a world-wide health burden by the year 2020. Inhaled anti-cholinergic therapy is currently considered the “gold standard” as first line therapy for COPD (Pauwels et al. 2001 Am. J. Respir. Crit. Care Med. 163:1256-1276).

Despite the large body of evidence supporting the use of anti-cholinergic therapy for the treatment of airway hyper reactive diseases, relatively few anti-cholinergic compounds are available for use in the clinic for pulmonary indications. More specifically, in United States, Ipratropium Bromide (Atrovent©; and Combivent©, in combination with albuterol) is currently the only inhaled anti-cholinergic marketed for the treatment of airway hyperreactive diseases. While this compound is a potent anti-muscarinic agent, it is short acting, and thus must be administered as many as four times daily in order to provide relief for the COPD patient. In Europe and Asia, the long-acting anti-cholinergic Tiotropium Bromide (Spiriva©) was recently approved, however this product is currently not available in the United States. Thus, there remains a need for novel compounds that are capable of causing blockade at mAChRs which are long acting and can be administered once-daily for the treatment of airway hyperreactive diseases such as asthma and COPD.

Since mAChRs are widely distributed throughout the body, the ability to apply anti-cholinergics locally and/or topically to the respiratory tract is particularly advantageous, as it would allow for lower doses of the drug to be utilized. Furthermore, the ability to design topically active drugs that have long duration of action, and in particular, are retained either at the receptor or by the lung, would allow the avoidance of unwanted side effects that may be seen with systemic anti-cholinergic use.

### SUMMARY OF THE INVENTION

This invention provides for a method of treating a muscarinic acetylcholine receptor (mAChR) mediated disease, wherein acetylcholine binds to an mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent.

Compounds of Formula (I) useful in the present invention are represented by the structure:

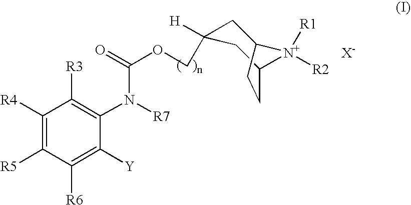

wherein:
Y is selected from the group consisting of

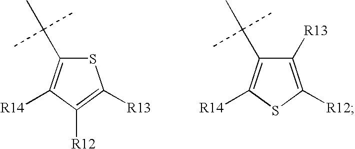

R1 and R2 are, independently, selected from the group consisting of a bond, hydrogen, and C1-4 alkyl;
R3, R4, R5 and R6 are, independently, selected from the group consisting of hydrogen, halogen, nitro, cyano, C1-10 alkyl, C2-10 alkenyl, C1-10 alkoxy, halosubstituted C1-10 alkoxy, (CR8R8)qORa, hydroxy, hydroxy substituted C1-4 alkyl (CR8R8)qNR10R11, (CR8R8)qNC(O)R9, and (CR8R8)qC(O)NR10R11; or two of either R3, R4, R5 or R6 moieties together may form a 5 to 6 membered saturated or unsaturated ring; and wherein the alkyl, aryl, arylalkyl, heteroaryl, heteroalkyl, heterocyclic or heterocyclicalkyl groups may be optionally substituted;
R12, R13 and R14 are independently selected from the group consisting of hydrogen, halogen, nitro, cyano, C1-4 alkyl, C2-4 alkenyl, C1-4 alkoxy, halosubstituted C1-4 alkoxy, (CR8R8)pORa, hydroxy, hydroxy substituted C1-4 alkyl, (CR8R8)pNR10 R11, (CR8RS)pNC(O)R9, and (CR8R8)pC(O)NR10R11; or two of either R12, R13 or R14 moieties together may form a 5 to 6 membered saturated or unsaturated ring; and wherein the alkyl, aryl, arylalkyl, heteroaryl, heteroalkyl, heterocyclic, or heterocyclicalkyl groups may be optionally substituted;
R7 is selected from the group consisting of hydrogen, and C1-4 alkyl;
R8 is hydrogen or C1-4 alkyl;
R9 is selected from the group consisting of hydrogen, optionally substituted C1-4 alkyl, and optionally substituted aryl;
R10 and R11 are, independently, selected from the group consisting of hydrogen, optionally substituted C1-4 alkyl, optionally substituted aryl, optionally substituted aryl C1-4 alkyl, optionally substituted heteroaryl, optionally substituted aryl C1-4 alkyl, optionally substituted heteroaryl, optionally substituted heteroaryl C1-4 alkyl, heterocyclic, and heterocyclic C1-4 alkyl; or R10 and R11 together with the nitrogen to which they are attached form a 5 to 7 membered ring which may optionally comprise an additional heteroatom selected from O, N and S;
Ra is selected from the group consisting of hydrogen, alkyl, aryl, aryl C1-4 alkyl, heteroaryl, heteroaryl C1-4 alkyl, heterocyclic and a heterocyclic C1-4 alkyl moiety, all of which moieties may be optionally substituted;
q is 0, or an integer having a value of 1 to 10;
p is 0, or an integer having a value of 1 to 4;
n is 0 or 1;
X- is a physiologically acceptable anion, such as chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

### DETAILED DESCRIPTION OF THE INVENTION

This invention related to novel bi-aryl 8-azoniabicyclo[3.2.1]octane compounds, pharmaceutical compositions, processes for their preparation, and use thereof in treating mAChR mediated diseases.

In a preferred embodiment of the present invention the compound is of formula (I) herein below:

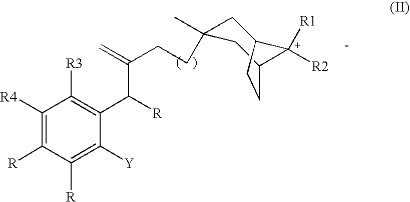

wherein:
Y is selected from the group consisting of

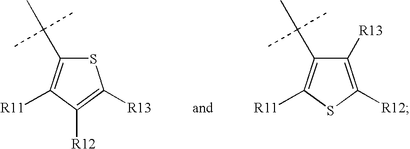

R1 and R2 are, independently, selected from the group consisting of a bond, hydrogen, and C1-4 alkyl;
R3, R4, R5 and R6 are, independently, selected from the group consisting of hydrogen, halogen, C1-5 alkyl, and C1-5 alkoxy;
R12, R13 and R14 are, independently, selected from the group consisting of hydrogen, halogen, and C1-4 alkyl;
R7 is selected from the group consisting of hydrogen, and methyl;
n is 0 or 1;
X- is a physiologically acceptable anion, such as chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

All of the aryl, heteroaryl, and heterocyclic containing moieties may be optionally substituted as defined herein below.

For use herein the term “the aryl, heteroaryl, and heterocyclic containing moieties” refers to both the ring and the alkyl, or if included, the alkenyl rings, such as aryl, arylalkyl, and aryl alkenyl rings. The term “moieties” and “rings” may be interchangeably used throughout.

As used herein, “optionally substituted” unless specifically defined shall mean such groups as halogen, such as fluorine, chlorine, bromine or iodine; hydroxy; hydroxy substituted C₁₋₁₀alkyl; C₁₋₁₀ alkoxy, such as methoxy or ethoxy; S(O)_{m′}C₁₋₁₀ alkyl, wherein m′ is 0, 1 or 2, such as methyl thio, methyl sulfinyl or methyl sulfonyl; amino, mono & di-substituted amino, such as in the NR₁₀R₁₁ group; NHC(O)R₉; C(O)NR₁₀R₁₁; C(O)OH; S(O)₂NR₁₀R₁₁; NHS(O)₂R₉, C₁₋₁₀ alkyl, such as methyl, ethyl, propyl, isopropyl, or t-butyl; halosubstituted C₁₋₁₀ alkyl, such CF₃; an optionally substituted aryl, such as phenyl, or an optionally substituted arylalkyl, such as benzyl or phenethyl, optionally substituted heterocylic, optionally substituted heterocyclicalkyl, optionally substituted heteroaryl, optionally substituted heteroaryl alkyl, wherein these aryl , heteroaryl, or heterocyclic moieties may be substituted one to two times by halogen; hydroxy; hydroxy substituted alkyl; C₁₋₁₀ alkoxy; S(O)m′C₁₋₁₀ alkyl; amino, mono & di-substituted alkyl amino, such as in the NR₁₀R₁₁ group; C₁₋₁₀ allkyl, or halosubstituted C₁₋₁₀ alkyl, such as CF₃.

The following terms, as used herein, refer to:
“halo”—all halogens, that is chloro, fluoro, bromo and iodo.
“C₁₋₁₀alkyl” or “alkyl”—both straight and branched chain moieties of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.
“cycloalkyl” is used herein to mean cyclic moiety, preferably of 3 to 8 carbons, including but not limited to cyclopropyl, cyclopentyl, cyclohexyl, and the like.
“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl- 1-propenyl, 1-butenyl, 2-butenyl and the like.
“aryl”—phenyl and naphthyl;
“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, tetrazole, thiazole, thiadiazole, triazole, imidazole, or benzimidazole.
“heterocyclic” (on its own or in any combination, such as “heterocyclicalkyl”)—a saturated or partially unsaturated 4-10 membered ring system in which one or more rings contain one or more heteroatoms selected from the group consisting of N, O, or S; such as, but not limited to, pyrrolidine, piperidine, piperazine, morpholine, tetrahydropyran, thiomorpholine, or imidazolidine. Furthermore, sulfur may be optionally oxidized to the sulfone or the sulfoxide.
“arylalkyl” or “heteroarylalkyl” or “heterocyclicalkyl” is used herein to mean C₁₋₁₀ alkyl, as defined above, attached to an aryl, heteroaryl or heterocyclic moiety, as also defined herein, unless otherwise indicated.
“sulfinyl”—the oxide S (O) of the corresponding sulfide, the term “thio” refers to the sulfide, and the term “sufonyl” refers to the fully oxidized S(O)₂ moiety.
“wherein two R₁ moieties (or two Y moieties) may together form a 5 or 6 membered saturated or unsaturated ring” is used herein to mean the formation of an aromatic ring system, such as naphthalene, or is a phenyl moiety having attached a 6 membered partially saturated or unsaturated ring such as a C₆ cycloalkenyl, i.e. hexene, or a C₅ cycloalkenyl moiety, such as cyclopentene.

Illustrative compounds of Formula (I) include, but are not limited to:
(3-endo)-8-azabicyclo[3.2.1]oct-3-ylmethyl[2-(3-thienyl)phenyl]carbamate;
(3-endo)-8-azabicyclo[3.2.1]oct-3-ylmethyl[5-chloro-2-(3-thienyl)phenyl]carbamate;
(3-endo)-8-azabicyclo[3.2.1]oct-3-ylmethyl[5-(methyloxy)-2-(3-thienyl)phenyl]carbamate;
(3-endo)-8-azabicyclo[3.2.1]oct-3-ylmethyl[4-methyl-2-(3-thienyl)phenyl]carbamate;
(3-endo)-8-azabicyclo[3.2.1]oct-3-ylmethyl[2-methyl-6-(3-thienyl)phenyl]carbamate;
(3-endo)-8-azabicyclo[3.2.1]oct-3-ylmethyl[3-fluoro-2-(3-thienyl)phenyl]carbamate;
(3-endo)-8-azabicyclo[3.2.1]oct-3-ylmethyl[4-fluoro-2-(3-thienyl)phenyl]carbamate;
(3-endo)-8-azabicyclo[3.2.1]oct-3-ylmethyl[5-methyl-2-(3-thienyl)phenyl]carbamate trifluoroacetate;
(3-endo)-8-azabicyclo[3.2.1]oct-3-ylmethyl[4-methyl-2-(3-thienyl)phenyl]carbamate trifluoroacetate;
(3-endo)-8-azabicyclo[3.2.1]oct-3-ylmethyl[5-fluoro-2-(3-thienyl)phenyl]carbamate trifluoroacetate;
(3-endo)-3-{[({[4-fluoro-2-(3-thienyl)phenyl]amino}carbonyl)oxy]methyl}-8,8-dimethyl-8-azoniabicyclo[3.2.1]octane trifluoroacetate;
(3-endo)-3-[({[4-fluoro-2-(3-thienyl)phenyl]amino}carbonyl)oxy]-8,8-dimethyl-8-azoniabicyclo[3.2.1]octane bromide;
(3-endo)-8,8-dimethyl-3-[({[2-(3-thienyl)phenyl]amino}carbonyl)oxy]-8-azoniabicyclo[3.2.1]octane bromide;
(3-endo)-3-[({[2-fluoro-6-(3-thienyl)phenyl]amino}carbonyl)oxy]-8,8-dimethyl-8-azoniabicyclo[3.2.1]octane bromide;
(3-endo)-3-[({[4-fluoro-2-(2-thienyl)phenyl]amino}carbonyl)oxy]-8,8-dimethyl-8-azoniabicyclo[3.2.1]octane bromide;
(3-endo)-8,8-dimethyl-3-[({[5-methyl-2-(3-thienyl)phenyl]amino }carbonyl)oxy]-8-azoniabicyclo[3.2.1]octane bromide;
(3-endo)-8,8-dimethyl-3-[({[4-methyl-2-(3-thienyl)phenyl]amino}carbonyl)oxy]-8-azoniabicyclo[3.2.1]octane bromide;
(3-endo)-3-[({[5-fluoro-2-(3-thienyl)phenyl]amino}carbonyl)oxy]-8,8-dimethyl-8-azoniabicyclo[3.2.1]octane bromide;
(3-endo)-8,8-dimethyl-3-[({[2-(2-thienyl)phenyl]amino}carbonyl)oxy]-8-azoniabicyclo[3.2.1]octane bromide;
(3-endo)-3-[({[4-chloro-2-(2-thienyl)phenyl]amino}carbonyl)oxy]-8,8-dimethyl-8-azoniabicyclo[3.2.1]octane bromide;
(3-endo)-8-methyl-8-azabicyclo[3.2.1]oct-3-yl[2-(3-thienyl)phenyl]carbamate;
(3-endo)-8,8-dimethyl-3-[({[5-methyl-2-(2-thienyl)phenyl]amino}carbonyl)oxy]-8-azoniabicyclo[3.2.1]octane bromide;
(3-endo)-8-methyl-8-azabicyclo[3.2.1]oct-3-yl[4-fluoro-2-(3-thienyl)phenyl]carbamate;
(3-endo)-8-methyl-8-azabicyclo[3.2.1]oct-3-yl[2-fluoro-6-(3-thienyl)phenyl]carbamate;
(3-endo)-8-methyl-8-azabicyclo[3.2.1]oct-3-yl[5-methyl-2-(3-thienyl)phenyl]carbamate;
(3-endo)-8,8-dimethyl-3-[({[4-methyl-2-(2-thienyl)phenyl]amino}carbonyl)oxy]-8-azoniabicyclo[3.2.1]octane bromide;
(3-endo)-8-methyl-8-azabicyclo[3.2.1]oct-3-yl[4-chloro-2-(3-thienyl)phenyl]carbamate;
(3-endo)-8-methyl-8-azabicyclo[3.2.1]oct-3-yl[4-methyl-2-(3-thienyl)phenyl]carbamate;
(3-endo)-8-methyl-8-azabicyclo[3.2.1]oct-3-yl[5-fluoro-2-(3-thienyl)phenyl]carbamate;
(3-endo)-3-[({[3-fluoro-2-(3-thienyl)phenyl]amino}carbonyl)oxy]-8,8-dimethyl-8-azoniabicyclo[3.2.1]octane bromide;
(3-endo)-8-methyl-8-azabicyclo[3.2.1]oct-3-yl[3-fluoro-2-(3-thienyl)phenyl]carbamate;
(3-endo)-8-methyl-8-azabicyclo[3.2.1]oct-3-yl[2-methyl-6-(2-thienyl)phenyl]carbamate; and
(3-endo)-8,8-dimethyl-3-[({[2-methyl-6-(2-thienyl)phenyl]amino}carbonyl)oxy]-8-azoniabicyclo[3.2.1]octane bromide.

The more preferred compounds useful in the present invention include, but are not limited to:
(3-endo)-3-[({[4-fluoro-2-(3-thienyl)phenyl]amino}carbonyl)oxy]-8,8-dimethyl-8-azoniabicyclo[3.2.1]octane bromide;
(3-endo)-8,8-dimethyl-3-[({[2-(3-thienyl)phenyl]amino}carbonyl)oxy]-8-azoniabicyclo[3.2.1]octane bromide;
(3-endo)-3-[({[2-fluoro-6-(3-thienyl)phenyl]amino}carbonyl)oxy]-8,8-dimethyl-8-azoniabicyclo[3.2.1]octane bromide;
(3-endo)-3-[({[4-fluoro-2-(2-thienyl)phenyl]amino }carbonyl)oxy]-8,8-dimethyl-8-azoniabicyclo[3.2.1]octane bromide;
(3-endo)-8,8-dimethyl-3-[({[5-methyl-2-(3-thienyl)phenyl]amino }carbonyl)oxy]—8-azoniabicyclo[3.2.1]octane bromide;
(3-endo)-8,8-dimethyl-3-[({[4-methyl-2-(3-thienyl)phenyl]amino}carbonyl)oxy]-8-azoniabicyclo[3.2.1]octane bromide;
(3-endo)-3-[({[5-fluoro-2-(3-thienyl)phenyl]amino}carbonyl)oxy]-8,8-dimethyl-8-azoniabicyclo[3.2.1]octane bromide;
(3-endo)-8,8-dimethyl-3-[({[2-(2-thienyl)phenyl]amino}carbonyl)oxy]-8-azoniabicyclo[3.2.1]octane bromide;
(3-endo)-3-[({[4-chloro-2-(2-thienyl)phenyl]amino}carbonyl)oxy]-8,8-dimethyl-8-azoniabicyclo[3.2.1]octane bromide;
(3-endo)-8-methyl-8-azabicyclo[3.2.1]oct-3-yl[2-(3-thienyl)phenyl]carbamate;
(3-endo)-8,8-dimethyl-3-[({[5-methyl-2-(2-thienyl)phenyl]amino}carbonyl)oxy]-8-azoniabicyclo[3.2.1]octane bromide;
(3-endo)-8-methyl-8-azabicyclo[3.2.1]oct-3-yl[4-fluoro-2-(3-thienyl)phenyl]carbamate;
(3-endo)-8-methyl-8-azabicyclo[3.2.1]oct-3-yl[2-fluoro-6-(3-thienyl)phenyl]carbamate;
(3-endo)-8-methyl-8-azabicyclo[3.2.1]oct-3-yl[5-methyl-2-(3-thienyl)phenyl]carbamate;
(3-endo)-8,8-dimethyl-3-[({[4-methyl-2-(2-thienyl)phenyl]amino }carbonyl)oxy]-8-azoniabicyclo[3.2.1]octane bromide;
(3-endo)-8-methyl-8-azabicyclo[3.2.1]oct-3-yl[4-chloro-2-(3-thienyl)phenyl]carbamate;
(3-endo)-8-methyl-8-azabicyclo[3.2.1]oct-3-yl[4-methyl-2-(3-thienyl)phenyl]carbamate;
(3-endo)-8-methyl-8-azabicyclo[3.2.1]oct-3-yl[5-fluoro-2-(3-thienyl)phenyl]carbamate;
(3-endo)-3-[({[3-fluoro-2-(3-thienyl)phenyl]amino}carbonyl)oxy]-8,8-dimethyl-8-azoniabicyclo[3.2.1]octane bromide;
(3-endo)-8-azabicyclo[3.2.1]oct-3-ylmethyl[4-fluoro-2-(3-thienyl)phenyl]carbamate;
(3-endo)-3-{[({[4-fluoro-2-(3-thienyl)phenyl]amino}carbonyl)oxy]methyl}-8,8-dimethyl-8-azoniabicyclo[3.2.1]octane trifluoroacetate;
(3-endo)-8-azabicyclo[3.2.1]oct-3-ylmethyl[5-methyl-2-(3-thienyl)phenyl]carbamate trifluoroacetate;
(3-endo)-8-azabicyclo[3.2.1]oct-3-ylmethyl[5-fluoro-2-(3-thienyl)phenyl]carbamate trifluoroacetate; and
(3-endo)-8-azabicyclo[3.2.1]oct-3-ylmethyl[2-(3-thienyl)phenyl]carbamate.

### Methods of Preparation

The compounds of Formula (I) may be obtained by applying synthetic procedures, some of which are illustrated in the Schemes below. The synthesis provided for these Schemes is applicable for producing compounds of Formula (I) having a variety of different Rₓ groups (X=1 to 14) which are reacted, employing substituents which are suitable protected, to achieve compatibility with the reactions outlined herein. Subsequent deprotection, in those cases, then affords compounds of the nature generally disclosed. While the Schemes are shown with compounds only of Formula (I), this is merely for illustration purpose only.

### Scheme 1

As outlined in Scheme 1, the desired compounds of Formula (I) can be prepared via the Curtius reaction of a suitable substituted 2-bromo-benzoic acid 1 with the suitably protected [3.2.1] bicyclic alcohol 2 using standard reagents well known in the art such as the commercially available diphenylphosphoryl azide (DPPA) reagent. The intermediate 3 thus formed can be coupled to a suitable boronic acid 4 using standard methods well known in the art such as the Suzuki coupling with catalytic tetra is(triphenylphosphino)palladium (0) in dimethylformamide and water in a presence of a base such as sodium carbonate or triethylamine. Removal of the protecting group (PG) on 3 using standard conditions such as treatment with p-toluenesulfonic acid in acetonitrile in the case of a BOC protecting group gives compound 5 of Formula (I) (R1=R2=H).

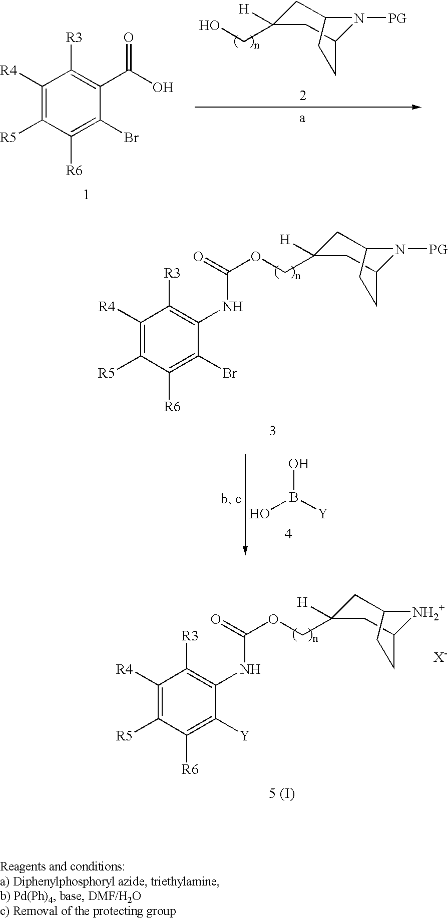

### Scheme 2

As shown in Scheme 2, the desired compounds of Formula (I) can also be prepared via the Curtius reaction of the suitable substituted 2-bromo-benzoic acid 1 with commercially available tropine 6 using standard reagents well known in the art such as the commercially available diphenylphosphoryl azide (DPPA) reagent. The intermediate 7 thus formed can be coupled to a suitable boronic acid 4 using standard methods well known in the art such as the Suzuki coupling with catalytic tetrakis(triphenylphosphino)palladium (0) in dimethylformamide in a presence of a base such as potassium carbonate to give compound 5 of Formula (I) (n=0, R1=methyl, R2=nothing).

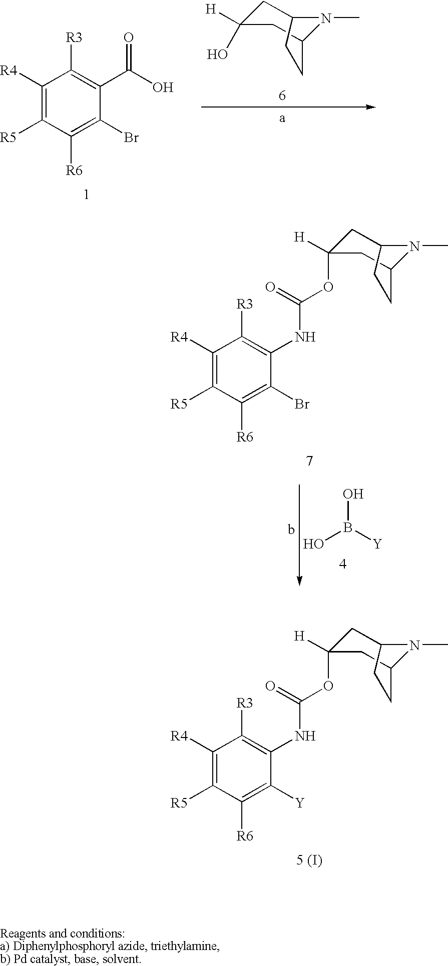

### Scheme 3

The required [3.2.1] bicyclic alcohol 2 (n=1, PG=BOC) is not commercially available but can be prepared from compound 8 which has been previously described in the literature (T. Momone et al, J. C. S. Perkin. Trans. 1, 9, 1997, 1307-14). As shown in Scheme 3, compound 9 was prepared by the Wittig reaction of compound 8 using standard reagents such as methyltriphenyl phosphonium bromide and potassium tert-butoxide. Hydroboration of the resulting alkene 9 with disiamylborane followed by oxidation produced the alcohol 10. Subsequent removal of the benzylic moiety of 10 under hydrogenation conditions followed by protection of the ring nitrogen with a BOC group using standard conditions such as treatment with di-tert-butyl dicarbonate in the presence of a base such as sodium hydroxide gave the desired alcohol 2 (n=1, PG=BOC).

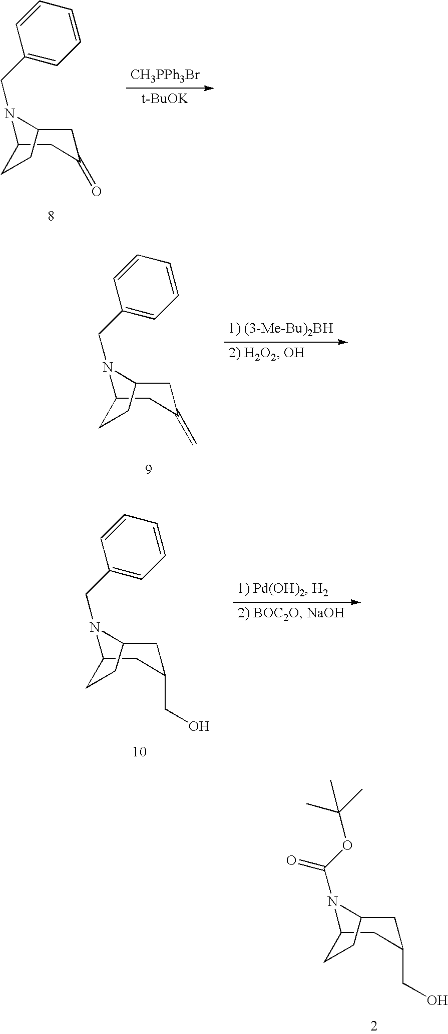

### Scheme 4

As outlined in Scheme 4, in the case where the compound of general formula (I) is a quaternary salt (R1 and R2 not H), it may be prepared by reacting sequentially the corresponding secondary or tertiary amine 5 (I, R1=H or methyl, R2=H or nothing) with suitable alkylating reagents (R1X and R2 X, R1 and R2 not H) in an inert solvent such as acetonitrile or dichloromethane.

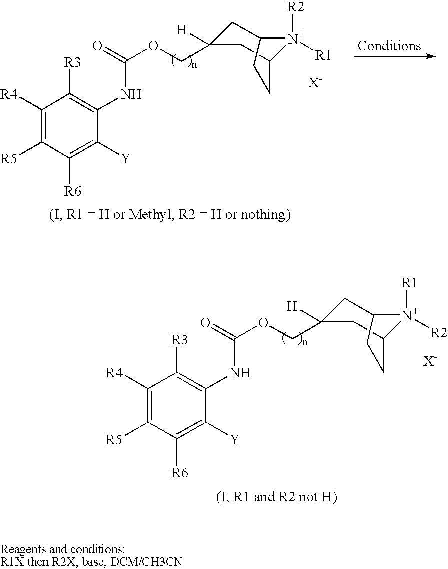

### SYNTHETIC EXAMPLES

The invention will now be described by reference to the following Examples, which are merely illustrative and are not to be construed as a limitation of the scope of the present invention. All temperatures are given in ° C. Thin layer chromatography (t.l.c.) was carried out on silica, and column chromatography on silica (Flash column chromatography using Merck 9385 unless stated otherwise).

LC/MS was conducted under the conditions described in System A or in System B:

### System A:

Column: 3.3 cm×4.6 mm ID, 3 um ABZ+PLUS
Flow Rate: 3 ml/min
Injection Volume: 5 μl
Temp: Room temperature
Solvents: A: 0.1% Formic Acid+10 mMolar Ammonium Acetate.
B: 95% Acetonitrile+0.05% Formic Acid
Gradient:

| Time | A % | B % |
|---|---|---|
| 0.00 | 100 | 0 |
| 0.70 | 100 | 0 |
| 4.20 | 0 | 100 |
| 5.30 | 0 | 100 |
| 5.50 | 100 | 0 |

### System B:

Liquid Chromatograph System: Shimadzu LC system with SCL-10A Controller and dual UV detector
Autosampler: Leap CTC with a Valco six port injector
Column: 1 mm×40 mm, Aquasil (C18)
Flow Rate: 0.3 mL/min
Injection Volume: 2 μl
Temp: room temperature
Solvents: A: 0.02% Trifluoroacetic Acid/Water.
B: 0.018% Trifluoroacetic Acid/Acetonitrile.
gradient (Linear):

| Time (min) | Duration (min) | A % | B % |
|---|---|---|---|
| 0.00 | 0.00 | 95 | 5 |
| 0.00 | 0.01 | 95 | 5 |
| 0.01 | 3.20 | 10 | 90 |
| 3.21 | 1.00 | 10 | 90 |
| 4.21 | 0.01 | 95 | 5 |
| 4.31 | 0.40 | 95 | 5 |

The Mass Directed Automated Preparative (MDAP) was conducted under the conditions described in System C or in System D:

### System C: Formate Salts

The preparative column used was a Supelcosil ABZplus (10 cm×2.12 cm internal diameter; particle size 5 m)
UV detection wavelength: 200-320 nM
Flow rate: 20 ml/min
Injection Volume: 0.5 ml
Solvent A: 0.1% formic acid
Solvent B: 95% acetonitrile+0.05% formic acid

### System D TFA Salts

The preparative column used was a Supelcosil ABZplus (10 cm×2.12 cm internal diameter; particle size 5 m)
UV detection wavelength: 200-320 nM
Flow rate: 20 ml/min
Injection Volume: 0.5 ml
Solvent A: water+0.1% trifluoroacetic acid
Solvent B: acetonitrile+0.1% trifluoroacetic acid
   Preparation of 1,1-dimethylethyl -(3-endo)-(hydroxymethyl)-8-azabicyclo[3.2.1]octane-8-carboxylate

The compound was prepared in three steps:

Step a: Preparation of 3-methylidene-8-(phenylmethyl)-8-azabicyclo[3.2.1]octane

A 500 ml flask with side arm, stirring bar, N₂ inlet, and septum stopper was charged with a solution of potassium tert-butoxide in THF (82 ml, 1M ) and methyltriphenyl phosphonium bromide (29.2 g, 82 mmol). It was cooled to 0° C. under dry N₂, and anhydrous THF (140 ml) was added via syringe at 0° C. The ylid solution was stirred for 20 min. 8-(Phenylmethyl)-8-azabicyclo[3.2.1]octan-3-one (14.0 g, 65 mmol) in anhydrous THF (ml) was added via syringe at 0° C. and the solution was stirred 1 h at room temperature then quenched with water (6 ml). The mixture was acidified to pH 1 and THF was removed in vacuo at 30° C. The residue was diluted with water (450 ml) and Ph₃PO was extracted with toluene (3×200 ml). The aqueous solution was basified with 6N NaOH (˜35 ml), and extracted with ethyl acetate (3×200 ml). The organic layers were combined, washed with saturated NaCl (3×100 ml), dried over Na₂SO₄, and evaporated to yield a crude product which was purified by flash chromatography (400 g of silica, ethyl acetate containing 0.1% TEA). 3-Methylidene-8-(phenylmethyl)-8-azabicyclo[3.2.1]octane was recovered as a yellow oil (11.3 g, 81.5%). LC/MS ESI R_{T} 1.27 min, MH⁺ 214.

NMR (CDCl₃, 400 MHz; δ): 1.58 ppm (q, 2H), 1.80-2.05 ppm (m, 4H), 2.55 ppm (d, 2H), 3.28 ppm (s, 2H), 3.65 ppm (s, 2H), 4.80 ppm (s, 2H), 7.29 ppm (t, 1H), 7.35 ppm (t, 2H), 7.46 ppm (d, 2H).

Step b: Preparation of (3-endo)-8-(phenylmethyl)-8-azabicyclo[3.2.1]oct-3-yl]methanol

A solution of disiamylborane was prepared by addition of 1.0 M borane in THF (20 ml, 20 mmol) to a 2.0 M solution of 2-methyl-2-butene in THF (20 ml, 40 mmol) at 0° C. under N₂. The solution was stirred 1 h at 0° C. before addition of 3-methylidene-8-(phenylmethyl)-8-azabicyclo[3.2.1]octane (1.07 g, 5 mmol) in 10 ml anhydrous THF. After 0.5 h at 0° C. the reaction mixture was warmed up to room temperature and allowed to stir overnight. The borane was quenched by careful addition of water (2 ml). The stirred solution was then oxidized at 0° C. by adding dropwise an aqueous solution of 30% H₂O₂ (3.87 ml, 45 mmol) over 30 minutes. The reaction mixture was neutralized with 3N HCl and the solvent was evaporated. The residue was taken up in ethyl acetate. Evaporation gave a viscous crude oil, which was used directly for step c.

Step c: Removal of the Benzyl Group and Protection with a BOC Group

A solution of (3-endo)-8-(phenylmethyl)-8-azabicyclo[3.2.1]oct-3-yl]methanol (1.16 g) (Schneider et al, Arch. Pharm., 1975, 308-365) in ethanol (20 ml) and 6N HCl (1 ml) containing palladium hydroxide on carbon (Pearlman's catalyst, 2.27 g, 22% (w/w)) was hydrogenated (55 psi H₂) at room temperature for 2 days. The catalyst was filtered off over Celite and the filtrate was evaporated under vacuum. The residue and di-tert-butyl dicarbonate (1.63 g, 7.5 mmol) were dissolved in 30 ml of dioxane: 1 N NaOH (2:1) and stirred overnight at room temperature. The solvent was evaporated and the residue partitioned between ethyl acetate (3×25 ml)and water (25 ml). The combined organic layers were dried over Na₂SO₄ and evaporated. The residue oil was purified by flash chromatography (150 g of silica, hexane:ethyl acetate (1:1, containing 0.1% 2.0 M NH₃ in methanol)). A colorless oil (0.65 g) was obtained. LC/MS ESI R_{T} 1.65 min, MH⁺ 242.

NMR (CDCl₃, 400 MHz; δ) 4.15 ppm (broad, 2H), 3.64 ppm (d, 2H), 2.20 ppm (broad, 2H),1.97 ppm (broad, 2H), 1.85 ppm (m, 1H), 1.60 ppm (m, 2H), 1.40-1.50 ppm (s+broad, 11H) .

Intermediate 1: 1,1-Dimethylethyl (3-endo)-[({[(2-bromo-5-methylphenyl)amino]carbonyl}oxy)methyl]-8-azabicyclo [3.2.1]octane-8-carboxylate

A solution of 2-bromo-5-methylbenzoic acid (430 mg) in chloroform (10 ml) was treated with diphenylphosphoryl azide (450 □l) and triethylamine (450 □l). The resulting reaction mixture was heated at 60° C. for 10 minutes then treated with a solution of 1,1-dimethylethyl -(3-endo)-(hydroxymethyl)-8-azabicyclo[3.2.1]octane-8-carboxylate (470 mg) in chloroform (2 ml). The reaction mixture was heated at reflux for 6 hours. The cooled solution was loaded onto a SPE cartridge (Si, 10 g). Elution with chloroform, followed by evaporation of the solvent gives the title compound (920 mg). LC/MS ESI R_{T} 3.93 mins MH⁺ 453.

Intermediate 2: 1,1-dimethylethyl (3-endo)-[({[(2-bromophenyl)amino]carbonyl}oxy)methyl]-8-azabicyclo[3.2.1]octane-8-carboxylate

The title compound was prepared from 2-bromobenzoic acid using the procedure described for the preparation of intermediate 1. LC/MS ESI R_{T} 3.79 mins MH⁺ 439.

Intermediate 3: 1,1-dimethylethyl (3-endo)-[({[(2-bromo-5-chlorophenyl)amino]carbonyl}oxy)methyl]-8-azabicyclo[3.2.1]octane-8-carboxylate

The title compound was prepared from 2-bromo-5-chlorobenzoic acid using the procedure described for the preparation of intermediate 1. LC/MS ESI R_{T} 4.05 mins MH⁺ 473.

Intermediate 4: 1,1-dimethylethyl (3-endo)-{[({[2-bromo-5-(methyloxy)phenyl]amino}carbonyl)oxy]methyl}-8-azabicyclo [3.2.1]octane-8-carboxylate

The title compound was prepared from 2-bromo-5-(methyloxy)benzoic acid using the procedure described for the preparation of intermediate 1. NMR (d⁶-DMSO 400 MHz; δ) 7.84 (br, 1H), 7.46-7.35 (m,1H), 7.18-7.11 (m,1H), 6.56-6.51 (m,1H), 4.33-4.10 (m's,4H), 3.81 (s,3H), 2.31-1.95 (m's,5H), 1.74-1.64 (m,2H), 1.5-1.41 (m's, 11H)

Intermediate 5: 1,1-dimethylethyl (3-endo)-[({[(2-bromo-4-methylphenyl)amino]carbonyl}oxy)methyl]-8-azabicyclo[3.2.1]octane-8-carboxylate

The title compound was prepared from 2-bromo-4-methylbenzoic acid using the procedure described for the preparation of intermediate 1. LC/MS ESI R_{T} 3.91 mins MH⁺ 453.

Intermediate 6: 1,1-dimethylethyl (3-endo)-[({[(2-bromo-6-methylyphenyl)amino]carbonyl}oxy)methyl]-8-azabicyclo[3.2.1]octane-8-carboxylate

The title compound was prepared from 2-bromo-6-methylbenzoic acid using the procedure described for the preparation of intermediate 1. LC/MS ESI R_{T} 3.64 mins MH⁺ 453.

Intermediate 7: 1,1-dimethylethyl (3-endo)-[({[(2-bromo-5-fluorophenyl)amino]carbonyl}oxy)methyl]-8-azabicyclo [3.2.1]octane-8-carboxylate

The title compound was prepared from 2-bromo-5-fluorobenzoic acid using the procedure described for the preparation of intermediate 1. LC/MS ESI R_{T} 3.92 mins M⁺ 457.

Intermediate 8: 1,1-dimethylethyl (3-endo)-[{[(2-bromo-3-fluorophenyl)amino]carbonyl}oxy)methyl]-8-azabicyclo[3.2.1]octane-8-carboxylate

The title compound was prepared from 2-bromo-3-fluorobenzoic acid using the procedure described for the preparation of intermediate 1. LC/MS ESI R_{T} 3.83 mins MH⁺ 457.

Intermediate 9: 1,1-dimethylethyl (3-endo)-[({[(2-bromo-4-fluorophenyl)amino]carbonyl}oxy)methyl]-8-azabicyclo [3.2.1]octane-8-carboxylate

The title compound was prepared from 2-bromo-4-fluorobenzoic acid using the procedure described for the preparation of intermediate 1. LC/MS ESI R_{T} 3.78 mins MH⁺ 457.

Intermediate 10: (3-endo)-8-methyl-8-azabicyclo[3.2.1]oct-3-yl (2-bromophenyl)carbamate

A solution of 2-bromobenzoic acid (1.00 g, 5.00 mmol) in THF (10 mL) was added to a Radleys® Carousel Reaction tube fitted with magnetic stirring bar. Diphenylphoshine azide (1.18 mL, 5.50 mmol) was then added, followed by ethylamine (1.40 mL, 10.0 mmol). The reaction mixture was stirred at room temperature for 10 minutes, and 8-methyl-8-azabicyclo[3.2.1]octan-3-ol (1.19 g, 7.50 mmol) was then added. Stirring continued for 16 h at 75° C., and the precipitated phosphonic acid was removed by vacuum filtration. The filtrate was then concentrated under reduced pressure. The residue was dissolved in DCM (6 ml), and the solution was transferred to a 10 mL hydrophobic frit that contained H₂O (3 mL). The aqueous layer was extracted with DCM (1×4 mL), and the combined organic layers were diluted with 50 mL of DCM. This solution was loaded onto a 10 g normal phase aminopropyl SPE cartridge primed with 60 mL of DCM. The cartridge was then sequentially eluted with DCM (1×60 mL), Et₂O (I×60 mL), EtOAc (5×60 mL), and MeOH (1×60 mL). The title compound was found in the EtOAc fractions, which were concentrated under reduced pressure to yield the title compound 0.309 g (18%). LC/MS ESI R_{T} 1.44 min, MH⁺ 339.

The following intermediates in Table 1 were prepared according to the procedure outlined for intermediate 10.

TABLE 1

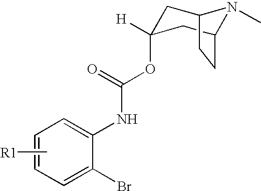

| Example | R1 | MS [M + H] | Rₜ (min) |
|---|---|---|---|
| 11 | 5-fluoro | 358 | 1.54 |
| 12 | 6-methyl | 354 | 1.32 |
| 13 | 4-methyl | 354 | 1.44 |
| 14 | 4-fluoro | 358 | 1.39 |
| 15 | 5-methyl | 354 | 1.46 |
| 16 | 5-chloro | 374 | 1.51 |
| 17 | 4-chloro | 374 | 1.43 |
| 18 | 6-fluoro | 358 | 1.28 |
| 19 | 3-fluoro | 358 | 1.45 |

### Example 1

(3-endo)-8-azabicyclo[3.2.1]oct-3-ylmethyl[2-(3-thienyl)phenyl]carbamate

A solution of and 1,1-dimethylethyl (3-endo)-[({[(2-bromophenyl)amino]carbonyl}oxy)methyl]-8-azabicyclo[3.2.1]octane-8-carboxylate (45 mg) and 3-thienylboronic acid (23.4 mg) in dimethylformamide (0.75 ml) was treated with sodium carbonate (30 mg), tetrakis(triphenylphosphino)palladium (0) (58 mg) and water (0.25 ml). The mixture was placed in a sealed reaction tube and heated in a microwave (CEM Explorer, 150° C., 10 minutes, pressure 250 psi, power 100 W). After cooling to room temperature, the solvent was removed under vacuum. The residue was dissolved in chloroform (1 ml) then washed sequentially with 2N hydrochloric acid (0.5 ml) and water (0.5 ml). The organic phase was separated and the solvent was removed under vacuum. The residue was dissolved in acetonitrile (1 ml) and treated with p-toluenesulfonic acid (20 mg). The resulting mixture was heated at reflux for 3 hours. After cooling to room temperature, the solution was purified by loading onto a SPE cartridge (SCX, 500 mg) then washing with methanol and eluting with 2M ammonia in methanol. The solvent was removed under vacuum and the residue was purified by MDAP to afford the title compound. LC/MS ESI R_{T} 2.18 mins MH⁺ 343

### Example 2

(3-endo)-8-azabicyclo[3.2.1]oct-3-ylmethyl[5-chloro-2-(3-thienyl)phenyl]carbamate

According to the procedure outlined in example 1, 3-thienylboronic acid and 1,1-dimethylethyl-(3-endo)-[({[(2-bromo-5-chlorophenyl)amino]carbonyl}oxy)methyl]-8-azabicyclo[3.2.1]octane-8-carboxylate were reacted to generate the title compound. LC/MS ESI R_{T} 2.67 mins MH⁺ 377.

### Example 3

(3-endo)-8-azabicyclo [3.2.1]oct-3-ylmethyl[5-(methyloxy)-2-(3-thienyl)phenyl]carbamate

According to the procedure outlined in example 1, 3-thienylboronic acid and 1,1-dimethylethyl (3-endo)-{[({[2-bromo-5-(methyloxy)phenyl]amino}carbonyl)oxy]methyl}-8-azabicyclo[3.2.1]octane-8-carboxylate were reacted to generate the title compound. LC/MS ESI R_{T} 2.51 mins MH⁺ 373

### Example 4

(3-endo)-8-azabicyclo [3.2.1]oct-3-ylmethyl[4-methyl-2-(3-thienyl)phenyl]carbamate

According to the procedure outlined in example 1, 3-thienylboronic acid and 1,1-dimethylethyl (3-endo)-[({[(2-bromo-4-methylphenyl)amino]carbonyl}oxy)methyl]-8-azabicyclo[3.2.1]octane-8-carboxylate were reacted to generate the title compound. LC/MS ESI R_{T} 2.54 mins MH⁺ 357.

### Example 5

(3-endo)-8-azabicyclo [3.2.1]oct-3-ylmethyl[2-methyl-6-(3-thienyl)phenyl]carbamate

According to the procedure outlined in example 1, 3-thienylboronic acid and 1,1-dimethylethyl (3-endo)-[({[(2-bromo-6-methylphenyl)amino]carbonyl}oxy)methyl]-8-azabicyclo[3.2.1]octane-8-carboxylate were reacted to generate the title compound. LC/MS ESI R_{T} 2.42 mins MH⁺ 357.

### Example 6

(3-endo)-8-azabicyclo[3.2.1]oct-3-ylmethyl[3-fluoro-2-(3-thienyl)phenyl]carbamate

According to the procedure outlined in example 1, 3-thienylboronic acid and 1,1-dimethylethyl (3-endo)-[({[(2-bromo-3-fluorophenyl)amino]carbonyl }oxy)methyl]-8-azabicyclo[3.2.1]octane-8-carboxylate were reacted to generate the title compound. LC/MS ESI R_{T} 2.51 mins MH⁺ 361.

### Example 7

(3-endo)-8-azabicyclo[3.2.1]oct-3-ylmethyl[4-fluoro-2-(3-thienyl)phenyl]carbamate

According to the procedure outlined in example 1, 3-thienylboronic acid and 1,1-dimethylethyl (3-endo)-[({[(2-bromo-4-fluorophenyl)amino]carbonyl}oxy)methyl]-8-azabicyclo[3.2.1]octane-8-carboxylate were reacted to generate the title compound. LCMS ESI R_{T} 2.46 mins MH⁺ 361.

### Example 8

(3-endo)-8-azabicyclo[3.2.1]oct-3-ylmethyl[5-methyl-2-(3-thienyl)phenyl]carbamate trifluoroacetate

A solution of 1,1-dimethylethyl (3-endo)-[({[(2-bromo-5-methylphenyl)amino]carbonyl}oxy)methyl]-8-azabicyclo[3.2.1]octane-8-carboxylate (45 mg) and 3-thienylboronic acid (21 mg) in dimethylformamide (0.75 ml) was treated with triethylamine (42 ul), tetrakis(triphenylphosphino)palladium (0) (58 mg) and water (0.25 ml). The mixture was placed in a sealed reaction tube and heated in a microwave (CEM Explorer, 150° C., 10 minutes, pressure 250 psi, power 100 W). After cooling to room temperature, the solvent was removed under vacuum. The residue was dissolved in chloroform (1 ml) then washed sequentially with 2N hydrochloric acid (0.5 ml) and water (0.5 ml). The organic phase was separated and the solvent was removed under vacuum. The residue was dissolved in acetonitrile (1 ml) and treated with p-toluenesulfonic acid (20 mg) . The resulting mixture was heated at reflux for 3 hours. After cooling to room temperature, the solution was purified by loading onto a SPE cartridge (SCX, 500 mg) then washing with methanol and eluting with 2M ammonia in methanol. The solvent was removed under vacuum and the residue was purified by MDAP to afford the title compound. LC/MS ESI R_{T} 2.54 mins MH⁺ 357.

### Example 9

(3-endo)-8-azabicyclo[3.2.1]oct-3-ylmethyl[5-fluoro-2-(3-thienyl)phenyl]carbamate trifluoroacetate

According to the procedure outlined in example 8, 3-thienylboronic acid and 1,1-dimethylethyl (3-endo)-[({[(2-bromo-5-fluorophenyl)amino]carbonyl}oxy)methyl]-8-azabicyclo[3.2.1]octane-8-carboxylate were reacted to generate the title compound. LC/MS ESI R_{T} 2.53 mins MH⁺ 361.

### Example 10: (3-endo)-3-{[({[4-fluoro-2-(3-thienyl)phenyl]amino}carbonyl)oxy]methyl}-8,8-dimethyl-8-azoniabicyclo [3.2.1]octane trifluoroacetate

A solution of (3-endo)-8-azabicyclo[3.2.1]oct-3-ylmethyl[4-fluoro-2-(3-thienyl)phenyl]carbamate (26 mg) in DMF (1 ml) was treated with methyl iodide (18 ul). After 16 h the solvent was evaporated and the residue purified by MDAP to give the title compound (8 mg). LC/MS ESI R_{T} 2.32 mins MH⁺ 389.

### Example 11

(3-endo)-8-methyl-8-azabicyclo[3.2.1]oct-3-yl[2-(3-thienyl)phenyl]carbamate

PS-PPh₃-Pd (0.020 g, 0.0026 mmol) was added to a solution of 8-methyl-8-azabicyclo[3.2.1]oct-3-yl (2-bromophenyl)carbamate (0.060 g, 0.177 mmol) in DME (1 mL) in a microwave reactor tube. A solution of 3-thiophene boronic acid (0.045 g, 0.354 mmol) in EtOH (1 mL) was added to the reaction mixture, followed by a solution of K₂CO₃ (0.056 g, 0.407 mmol) in H₂O (0.5 mL). The reaction vial was capped and heated at 165° C. for 10 min. The resin was removed by gravity filtration, washed with DME (1 mL) and EtOH (1 mL), and the filtrate was concentrated under reduced pressure. The residue was dissolved in DCM (4 mL) and transferred onto a 6 mL hydrophobic frit. H₂O (2.0 mL) was added to the solution and mixed to remove base. The layers were separated, and the aqueous layer was washed with DCM (1×4 mL). The combined organic layers were concentrated under reduced pressure and purified by Gilson® preparatory HPLC to yield the title compound (36.4 mg, 61%). LC/MS ESI R_{T} 1.41 min, MH⁺ 343.

The following examples in Table 2 were prepared according to the procedure outlined in Example 11.

TABLE 2

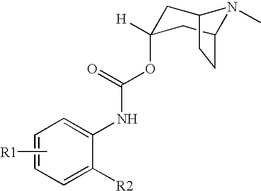

| | | | MS | |
|---|---|---|---|---|
| Example | R1 | R2 | [M + H] | Rₜ (min) |
| 12 | 4-fluoro | 3-thiophene | 361 | 1.49 |
| 13 | 5-fluoro | 3-thiophene | 360 | 1.58 |
| 14 | 4-methyl | 3-thiophene | 358 | 1.64 |
| 15 | 5-methyl | 3-thiophene | 357 | 1.62 |
| 16 | 4-chloro | 3-thiophene | 377 | 1.74 |

### Example 17

(3-endo)-8-methyl-8-azabicyclo [3.2.1]oct-3-yl[3-fluoro-2-(3-thienyl)phenyl]carbamate

Pd(PPh₃)₄ (0.075 g, 0.065 mmol) was added to a solution of 8-methyl-8-azabicyclo[3.2.1]oct-3-yl (2-bromo-3-fluorophenyl)carbamate (0.116 g, 0.324 mmol) in DME (1 mL) in a 4 mL glass vial with a magnetic stir bar. A solution of 3-thiophene boronic acid (0.083 g, 0.648 mmol) in EtOH (1 mL) was added to the reaction mixture, followed by a solution of K₂CO₃ (0.168 g, 1.22 mmol) in H₂O (0.5 mL). The glass vial was capped and heated at 80° C. for 16 h. The reaction mixture was concentrated under reduced pressure, taken up in DCM (4 mL), and transferred onto a 6 mL hydrophobic frit. H₂O (2 mL) was added to the solution and mixed to remove base. The layers were separated, and the aqueous layer was washed with DCM (1×4 mL). The combined organic layers were concentrated under reduced pressure and purified by Gilson® preparatory BPLC to the title compound (0.042 g, 36%). LC/MS ESI R_{T} 1.56 min, MH⁺ 359.

The following examples in Table 3 were prepared according to the procedure outlined in Example 17.

TABLE 3

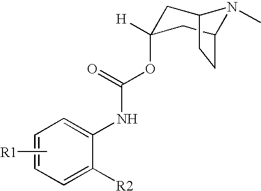

| | | | MS | |
|---|---|---|---|---|
| Example | R1 | R2 | [M + H] | Rₜ (min) |
| 18 | 6-fluoro | 3-thiophene | 361 | 1.35 |
| 19 | 6-methyl | 2-thiophene | 357 | 1.13 |

### Example 20

(3-endo)-8,8-dimethyl-3-[({[2-methyl-6-(2-thienyl)phenyl]amino}carbonyl)oxy]-8-azoniabicyclo [3.2.1]octane

First Step

Pd(PPh₃)₄ (0.046 g, 0.040 mmol) was added to a solution of 8-methyl-8-azabicyclo[3.2.1]oct-3-yl (2-bromo-6-methylphenyl)carbamate (0.071 g, 0.200 mmol) in DME (1 mL) in a 4 mL glass vial with a magnetic stir bar. A solution of 2-thiophene boronic acid (0.038 g, 0.300 mmol) in EtOH (1 mL) was added to the reaction mixture, followed by a solution of K₂CO₃ (0.064 g, 0.460 mmol) in H₂O (0.5 mL). The glass vial was capped and heated at 80° C. for 16 h. The reaction mixture was concentrated under reduced pressure, taken up in DCM (4 mL), and transferred onto a 6 mL hydrophobic frit. H₂O (2 mL) was added to the solution and mixed to remove base. The layers were separated, and the aqueous layer was washed with DCM (1×4 mL). The combined organic layers were concentrated under reduced pressure and purified by Gilson® preparatory HPLC to yield 8-methyl-8-azabicyclo[3.2.1]oct-3-yl[2-methyl-6-(2-thienyl)phenyl]carbamate (0.048 g, 67%). LC/MS ESI R_{T} 1.13 min, MH⁺ 357.

Second Step

A 2 M solution of MeBr in tert-butyl methyl ether (0.700 mL, 1.49 mmol) was added to a solution of 8-methyl-8-azabicyclo[3.2.1]oct-3-yl[2-methyl-6-(2-thienyl)phenyl]carbamate (0.040 g, 0.112 mmol) in a mixture (1:1) of DCM/CH₃CN (2 mL) in a glass vial with a magnetic stirring bar under argon. The reaction was stirred at room temperature for 16 h. The solvent was evaporated, and the product was dried under high vacuum to yield the title compound (0.041 g, 99%). LC/MS ESI R_{T} 1.62 min, MH⁺ 371.

The following examples in Table 4 were prepared according to the procedure outlined in Example 20 reacting the appropriate boronic acid and bromo-phenyl intermediates.

TABLE 4

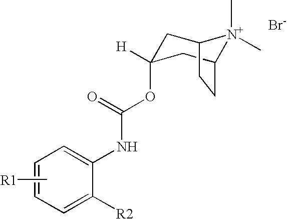

| Example | R1 | R2 | MS [M+] | Rₜ (min) |
|---|---|---|---|---|
| 21 | H | 3-thiophene | 357 | 1.20 |
| 22 | 3-fluoro | 3-thiophene | 375 | 1.55 |
| 23 | 4-fluoro | 2-thiophene | 375 | 1.66 |
| 24 | 4-chloro | 2-thiophene | 391 | 1.77 |
| 25 | 4-fluoro | 3-thiophene | 375 | 1.25 |
| 26 | 5-methyl | 2-thiophene | 371 | 1.74 |
| 27 | 5-fluoro | 3-thiophene | 375 | 1.37 |
| 28 | 6-fluoro | 3-thiophene | 375 | 1.44 |
| 29 | 4-methyl | 3-thiophene | 371 | 1.40 |
| 30 | 5-methyl | 3-thiophene | 371 | 1.69 |
| 31 | 4-methyl | 2-thiophene | 371 | 1.77 |
| 32 | H | 2-thiophene | 357 | 1.61 |

Abbreviations
BOC tert-butyloxycarbonyl
DCM Dichloromethane
DME Ethylene glycol dimethyl ether
DMF Dimethylformamide
ESI Electrospray ionization
HPLC High pressure liquid chromatography
LC/MS Liquid chromatography/Mass spectrometry
MDAP Mass directed automated preparative
Rt Retention time
SPE Solid phase extraction
TEA Triethylamine
TFA Trifluoroacetic acid
THF Tetrahydrofuran

### Biological Examples

The inhibitory effects of compounds at the M₃ mAChR of the present invention are determined by the following in vitro and in vivo functional assays:

### Analysis of Inhibition of Receptor Activation by Calcium Mobilization:

Stimulation of mAChRs expressed on CHO cells were analyzed by monitoring receptor-activated calcium mobilization as previously described (H. M. Sarau et al, 1999. Mol. Pharmacol. 56, 657-663). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 MM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels. The emitted fluorescence from all 96 wells is measured simultaneously using a cooled CCD camera. Data points are collected every second. This data was then plotting and analyzed using GraphPad PRISM software.

### Methacholine-Induced Bronchoconstriction—Potency and Duration of Action

Airway responsiveness to methacholine was determined in awake. unrestrained Balb C mice (n=6 each group). Barometric plethysmography was used to measure enhanced pause (Penh), a unitless measure that has been shown to correlate with the changes in airway resistance that occur during bronchial challenge with methacholine(2). Mice were pre-treated with 50 μl of compound (0.003-10 μg/mouse) in 50 μl of vehicle (10% DMSO) intranasally (i.n.) and were then placed in the plethysmography chamber a given amount of time following drug administration (15 min-96 h). For potency determination, a dose response to a given drug was performed, and all measurements were taken 15 min following i.n. drug administration. For duration of action determination, measurements were taken anywhere from 15 min to 96 hours following i.n. drug administration.

Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software. This experiment allows the determination of duration of activity of the administered compound.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis.

### Formulation-Administration

Accordingly, the present invention further provides a pharmaceutical formulation comprising a compound of formula (I), or a pharmaceutically acceptable salt, solvate, or physiologically functional derivative (e.g., salts and esters) thereof, and a pharmaceutically acceptable carrier or excipient, and optionally one or more other therapeutic ingredients.

Hereinafter, the term “active ingredient” means a compound of formula (I), or a pharmaceutically acceptable salt, solvate, or physiologically functional derivative thereof.

Compounds of formula (I) will be administered via inhalation via the mouth or nose.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g., lactose or starch), organic or inorganic salts (e.g., calcium chloride, calcium phosphate or sodium chloride), polyalcohols (e.g., mannitol), or mixtures thereof, alternatively with one or more additional materials, such additives included in the blend formulation to improve chemical and/or physical stability or performance of the formulation, as discussed below, or mixtures thereof. Use of lactose is preferred. Each capsule or cartridge may generally contain between 20 μg-10 mg of the compound of formula (I) optionally in combination with another therapeutically active ingredient. Alternatively, the compound of the invention may be presented without excipients, or may be formed into particles comprising the compound, optionally other therapeutically active materials, and excipient materials, such as by co-precipitation or coating.

Suitably, the medicament dispenser is of a type selected from the group to consisting of a reservoir dry powder inhaler (RDPI), a multi-dose dry powder inhaler (MDPI), and a metered dose inhaler (MDI).

By reservoir dry powder inhaler (RDPI) it is meant as an inhaler having a reservoir form pack suitable for comprising multiple (un-metered doses) of medicament in dry powder form and including means for metering medicament dose from the reservoir to a delivery position. The metering means may for example comprise a metering cup or perforated plate, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disk-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants e.g. oleic acid or lecithin and cosolvents e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum aerodynamic particle size for inhalation into the bronchial system for localized delivery to the lung is usually 1-10 μm, preferably 2-5μm. The optimum aerodynamic particle size for inhalation into the alveolar region for achieving systemic delivery to the lung is approximately 0.5-3 μm, preferably 1-3 μm. Particles having an aerodynamic size above 20 μm are generally too large when inhaled to reach the small airways. Average aerodynamic particle size of a formulation may be measured by, for example cascade impaction. Average geometric particle size may be measured, for example by laser diffraction, optical means.

To achieve a desired particle size, the particles of the active ingredient as produced may be size reduced by conventional means e.g. by controlled crystallization, micronisation or nanomilling. The desired fraction may be separated out by air classification. Alternatively, particles of the desired size may be directly produced, for example by spray drying, controlling the spray drying parameters to generate particles of the desired size range. Preferably, the particles will be crystalline, although amorphous material may also be employed where desirable. When an excipient such as lactose is employed, generally; the particle size of the excipient will be much greater than the inhaled medicament within the present invention, such that the “coarse” carrier is non-respirable. When the excipient is lactose it will typically be present as milled lactose, wherein not more than 85% of lactose particles will have a MMD of 60-90 μm and not less than 15% will have a MMD of less than 15 μm. Additive materials in a dry powder blend in addition to the carrier may be either respirable, i.e., aerodynamically less than 10 microns, or non-respirable, i.e., aerodynamically greater than 10 microns.

Suitable additive materials which may be employed include amino acids, such as leucine; water soluble or water insoluble, natural or synthetic surfactants, such as lecithin (e.g., soya lecithin) and solid state fatty acids (e.g., lauric, palmitic, and stearic acids) and derivatives thereof (such as salts and esters); phosphatidylcholines; sugar esters. Additive materials may also include colorants, taste masking agents (e.g., saccharine), anti-static-agents, lubricants (see, for example, Published PCT Patent Appl. No. WO 87/905213, the teachings of which are incorporated by reference herein), chemical stabilizers, buffers, preservatives, absorption enhancers, and other materials known to those of ordinary skill.

Sustained release coating materials (e.g., stearic acid or polymers, e.g. polyvinyl pyrolidone, polylactic acid) may also be employed on active material or active material containing particles (see, for example, U.S. Pat. No. 3,634,582, GB 1,230,087, GB 1,381,872, the teachings of which are incorporated by reference herein).

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing an effective dose, as herein before recited, or an appropriate fraction thereof, of the active ingredient.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claims is:

1. A compound according to formula (I) as indicated below 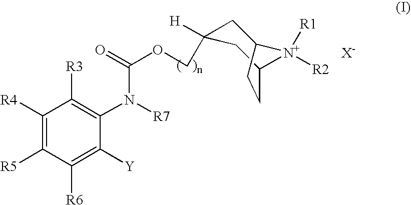
wherein:
Y is selected from the group consisting of 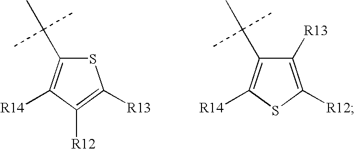
R1 and R2 are, independently, selected from the group consisting of a bond, hydrogen, and C1-4 alkyl;
R3, R4, R5 and R6 are, independently, selected from the group consisting of hydrogen, halogen, nitro, cyano, C1-C10 alkyl, C2-10 alkenyl, C1-10 alkoxy, halosubstituted C1-10 alkoxy, (CR8R8)qORa, hydroxy, hydroxy substituted C1-4 alkyl, (CR8R8)qNR10R11, (CR8R8)qNC(O)R9, and (CR8R8)qC(O)NR10R11; or two of either R3, R4, R5 or R6 moieties together may form a 5 to 6 membered saturated or unsaturated ring; and wherein the alkyl, aryl, arylalkyl, heteroaryl, heteroalkyl, heterocyclic or heterocyclicalkyl groups may be optionally substituted;
R12, R13 and R14 are independently selected from the group consisting of hydrogen, halogen, nitro, cyano, C1-4 alkyl, C2-4 alkenyl, C1-4 alkoxy, halosubstituted C1-4 alkoxy, (CR8R8)pORa, hydroxy, hydroxy substituted C1-4 alkyl, (CR8R8)pNR10R11, (CR8R8)pNC(O)R9, and (CR8R8)pC(O)NR10R11; or two of either R12, R13 or R14 moieties together may form a 5 to 6 membered saturated or unsaturated ring; and wherein the alkyl, aryl, arylalkyl, heteroaryl, heteroalkyl, heterocyclic, or heterocyclicalkyl groups may be optionally substituted;
R7 is selected from the group consisting of hydrogen, and C1-4 alkyl;
R8 is hydrogen or C1-4 alkyl;
R9 is selected from the group consisting of hydrogen, optionally substituted C1-4 alkyl, and optionally substituted aryl;
R10 and R11 are, independently, selected from the group consisting of hydrogen, optionally substituted C1-4 alkyl, optionally substituted aryl, optionally substituted aryl C1-4 alkyl, optionally substituted heteroaryl, optionally substituted aryl C 1-4 alkyl, optionally substituted heteroaryl, optionally substituted heteroaryl C 1-4 alkyl, heterocyclic, and heterocyclic C1-4 alkyl; or R10 and R11 together with the nitrogen to which they are attached form a 5 to 7 membered ring which may optionally comprise an additional heteroatom selected from O, N and S;
Ra is selected from the group consisting of hydrogen, alkyl, aryl, aryl C1-4 alkyl, heteroaryl, heteroaryl C1-4 alkyl, heterocyclic and a heterocyclic C1-4 alkyl moiety, all of which moieties may be optionally substituted;
q is 0, or an integer having a value of 1 to 10;
p is 0, or an integer having a value of 1 to 4;
n is 0 or 1;
X- is a physiologically acceptable anion, such as chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

2. A compound according to formula II herein below 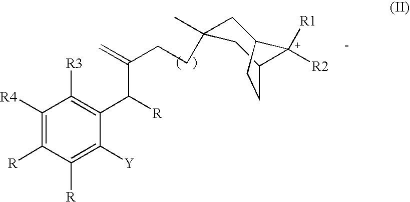
wherein:
Y is selected from the group consisting of 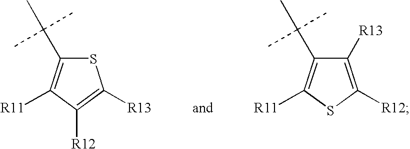
R1 and R2 are, independently, selected from the group consisting of a bond, hydrogen, and C1-4 alkyl;
R3, R4, R5 and R6 are, independently, selected from the group consisting of hydrogen, halogen, C1-5 alkyl, and C1-5 alkoxy;
R12, R13 and R14 are, independently, selected from the group consisting of hydrogen, halogen, and C1-4 alkyl;
R7 is selected from the group consisting of hydrogen, and methyl;
n is 0 or 1;
X- is a physiologically acceptable anion, such as chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

3. A compound according to claim 1 selected from the group of:
(3-endo)-8-azabicyclo[3.2.1]oct-3-ylmethyl[2-(3-thienyl)phenyl]carbamate;
(3-endo)-8-azabicyclo[3.2.1]oct-3-ylmethyl[5-chloro-2-(3-thienyl)phenyl]carbamate;
(3-endo)-8-azabicyclo[3.2.1]oct-3-ylmethyl[5-(methyloxy)-2-(3-thienyl)phenyl]carbamate;
(3-endo)-8-azabicyclo[3.2.1]oct-3-ylmethyl[4-methyl-2-(3-thienyl)phenyl]carbamate;
(3-endo)-8-azabicyclo[3.2.1]oct-3-ylmethyl[2-methyl-6-(3-thienyl)phenyl]carbamate;
(3-endo)-8-azabicyclo[3.2.1]oct-3-ylmethyl[3-fluoro-2-(3-thienyl)phenyl]carbamate;
(3-endo)-8-azabicyclo[3.2.1]oct-3-ylmethyl[4-fluoro-2-(3-thienyl)phenyl]carbamate;
(3-endo)-8-azabicyclo[3.2.1]oct-3-ylmethyl[5-methyl-2-(3-thienyl)phenyl]carbamate trifluoroacetate;
(3-endo)-8-azabicyclo[3.2.1]oct-3-ylmethyl[4-methyl-2-(3-thienyl)phenyl]carbamate trifluoroacetate;
(3-endo)-8-azabicyclo[3.2.1]oct-3-ylmethyl[5-fluoro-2-(3-thienyl)phenyl]carbamate trifluoroacetate;
(3-endo)-3-{[({[4-fluoro-2-(3-thienyl)phenyl]amino}carbonyl)oxy]methyl}8,8-dimethyl-8-azoniabicyclo[3.2.1]octane trifluoroacetate;
(3-endo)-3-[({[4-fluoro-2-(3-thienyl)phenyl]amino}carbonyl)oxy]-8,8-dimethyl-8-azoniabicyclo[3.2.1]octane bromide;
(3-endo)-8,8-dimethyl-3-[({[2-(3-thienyl)phenyl]amino}carbonyl)oxy]-8-azoniabicyclo[3.2.1]octane bromide;
(3-endo)-3-[({[2-fluoro-6-(3-thienyl)phenyl]amino}carbonyl)oxy]-8,8-dimethyl-8-azoniabicyclo[3.2.1]octane bromide;
(3-endo)-3-[({[4-fluoro-2-(2-thienyl)phenyl]amino}carbonyl)oxy]-8,8-dimethyl-8-azoniabicyclo[3.2.1]octane bromide;
(3-endo)-8,8-dimethyl-3-[({[5-methyl-2-(3-thienyl)phenyl]amino}carbonyl)oxy]-8-azoniabicyclo[3.2.1]octane bromide;
(3-endo)-8,8-dimethyl-3-[({[4-methyl-2-(3-thienyl)phenyl]amino}carbonyl)oxy]-8-azoniabicyclo[3.2.1]octane bromide;
(3-endo)-3-[({[5-fluoro-2-(3-thienyl)phenyl]amino }carbonyl)oxy]-8,8-dimethyl-8-azoniabicyclo[3.2.1]octane bromide;
(3-endo)-8,8-dimethyl-3-[({[2-(2-thienyl)phenyl]amino}carbonyl)oxy]-8-azoniabicyclo[3.2.1]octane bromide;
(3-endo)-3-[({[4-chloro-2-(2-thienyl)phenyl]amino}carbonyl)oxy]-8,8-dimethyl-8-azoniabicyclo[3.2.1]octane bromide;
(3-endo)-8-methyl-8-azabicyclo[3.2.1]oct-3-yl[2-(3-thienyl)phenyl]carbamate;
(3-endo)-8,8-dimethyl-3-[({[5-methyl-2-(2-thienyl)phenyl]amino}carbonyl)oxy]-8-azoniabicyclo[3.2.1]octane bromide;
(3-endo)-8-methyl-8-azabicyclo[3.2.1]oct-3-yl[4-fluoro-2-(3-thienyl)phenyl]carbamate;
(3-endo)-8-methyl-8-azabicyclo[3.2.1]oct-3-yl[2-fluoro-6-(3-thienyl)phenyl]carbamate;
(3-endo)-8-methyl-8-azabicyclo[3.2.1]oct-3-yl[5-methyl-2-(3-thienyl)phenyl]carbamate;
(3-endo)-8,8-dimethyl-3-[({[4-methyl-2-(2-thienyl)phenyl]amino}carbonyl)oxy]-8-azoniabicyclo[3.2.1]octane bromide;
(3-endo)-8-methyl-8-azabicyclo[3.2.1]oct-3-yl[4-chloro-2-(3-thienyl)phenyl]carbamate;
(3-endo)-8-methyl-8-azabicyclo[3.2.1]oct-3-yl[4-methyl -2-(3-thienyl)phenyl]carbamate;
(3-endo)-8-methyl-8-azabicyclo[3.2.1]oct-3-yl[5-fluoro-2-(3-thienyl)phenyl]carbamate;
(3-endo)-3-[({[3-fluoro-2-(3-thienyl)phenyl]amino}carbonyl)oxy]-8,8-dimethyl8-azoniabicyclo[3.2.1]octane bromide;
(3-endo)-8-methyl-8-azabicyclo[3.2.1]oct-3-yl[3-fluoro-2-(3-thienyl)phenyl]carbamate;
(3-endo)-8-methyl-8-azabicyclo[3.2.1]oct-3-yl[2-methyl-6-(2-thienyl)phenyl]carbamate; and
(3-endo)-8,8-dimethyl-3-[({[2-methyl-6-(2-thienyl)phenyl]amino}carbonyl)oxy]-8-azoniabicyclo[3.2.1]octane bromide.

4 A compound according to claim 1 herein below
(3-endo)-3-[({[4-fluoro-2-(3-thienyl)phenyl]amino}carbonyl)oxy]-8,8-dimethyl-8-azoniabicyclo[3.2.1]octane bromide;
(3-endo)-8,8-dimethyl-3-[({[2-(3-thienyl)phenyl]amino}carbonyl)oxy]-8-azoniabicyclo[3.2.1]octane bromide;
(3-endo)-3-[({[2-fluoro-6-(3-thienyl)phenyl]amino}carbonyl)oxy]-8,8-dimethyl-8-azoniabicyclo[3.2.1]octane bromide;
(3-endo)-3-[({[4-fluoro-2-(2-thienyl)phenyl]amino}carbonyl)oxy]-8,8-dimethyl-8-azoniabicyclo[3.2.1]octane bromide;
(3-endo)-8,8-dimethyl-3-[({[5-methyl-2-(3-thienyl)phenyl]amino}carbonyl)oxy]-8-azoniabicyclo[3.2.1]octane bromide;
(3endo)-8,8-dimethyl-3-[({[4-methyl-2-(3-thienyl)phenyl]amino}carbonyl)oxy]-8-azoniabicyclo[3.2.1]octane bromide;
(3-endo)-3-[({[5-fluoro-2-(3-thienyl)phenyl]amino}carbonyl)oxy]-8,8-dimethyl-8-azoniabicyclo[3.2.1]octane bromide;
(3-endo)-8,8-dimethyl-3-[({[2-(2-thienyl)phenyl]amino}carbonyl)oxy]-8-azoniabicyclo[3.2.1]octane bromide;
(3-endo)-3-[({[4-chloro-2-(2-thienyl)phenyl]amino}carbonyl)oxy]-8,8-dimethyl-8-azoniabicyclo[3.2.1]octane bromide;
(3-endo)-8-methyl-8-azabicyclo[3.2.1]oct-3-yl[2-(3-thienyl)phenyl]carbamate;
(3-endo)-8,8-dimethyl-3-[({[5-methyl-2-(2-thienyl)phenyl]amino}carbonyl)oxy]-8-azoniabicyclo[3.2.1]octane bromide;
(3-endo)-8-methyl-8-azabicyclo[3.2.1]oct-3-yl[4-fluoro-2-(3-thienyl)phenyl]carbamate;
(3-endo)-8-methyl-8-azabicyclo[3.2.1]oct-3-yl[2-fluoro-6-(3-thienyl)phenyl]carbamate;
(3-endo)-8-methyl-8-azabicyclo[3.2.1]oct-3-yl[5-methyl-2-(3-thienyl)phenyl]carbamate;
(3-endo)-8,8-dimethyl-3-[({[4-methyl-2-(2-thienyl)phenyl]amino}carbonyl)oxy]-8-azoniabicyclo[3.2.1]octane bromide;
(3-endo)-8-methyl-8-azabicyclo[3.2.1]oct-3-yl[4-chloro-2-(3-thienyl)phenyl]carbamate;
(3-endo)-8-methyl-8-azabicyclo[3.2.1]oct-3-yl[4-methyl-2-(3-thienyl)phenyl]carbamate;
(3-endo)-8-methyl-8-azabicyclo[3.2.1]oct-3-yl[5-fluoro-2-(3-thienyl)phenyl]carbamate;
(3-endo)-3-[({[3-fluoro-2-(3-thienyl)phenyl]amino}carbonyl)oxy]-8,8-dimethyl-8-azoniabicyclo[3.2.1]octane bromide;
(3-endo)-8-azabicyclo [3.2.1]oct-3-ylmethyl[4-fluoro-2-(3-thienyl)phenyl]carbamate;
(3-endo)-3-{[({[4-fluoro-2-(3-thienyl)phenyl]amino}carbonyl)oxy]methyl}-8,8-dimethyl-8-azoniabicyclo[3.2.1]octane trifluoroacetate;
(3-endo)-8-azabicyclo [3.2.1]oct-3-ylmethyl[5-methyl-2-(3-thienyl)phenyl]carbamate trifluoroacetate;
(3-endo)-8-azabicyclo[3.2.1]oct-3-ylmethyl[5-fluoro-2-(3-thienyl)phenyl]carbamate trifluoroacetate; and
(3-endo)-8-azabicyclo [3.2.1]oct-3-ylmethyl[2-(3-thienyl)phenyl]carbamate.

5. A pharmaceutical composition for the treatment of muscarinic acetylcholine receptor mediated diseases comprising a compound according to claim 1 and a pharmaceutically acceptable carrier thereof.

6. A method of inhibiting the binding of acetylcholine to its receptors in a mammal in need thereof comprising administering a safe and effective amount of a compound according to claim 1.

7. A method of treating a muscarinic acetylcholine receptor mediated disease, wherein acetylcholine binds to said receptor, comprising administering a safe and effective amount of a compound according to claim 1.

8. A method according to claim 7 wherein the disease is selected from the group consisting of chronic obstructive lung disease, chronic bronchitis, asthma, chronic respiratory obstruction, pulmonary fibrosis, pulmonary emphysema and allergic rhinitis.

9. A method according to claim 7 wherein administration is via inhalation via the mouth or nose.

10. A method according to claim 7 wherein administration is via a medicament dispenser selected from a reservoir dry powder inhaler, a multi-dose dry powder inhaler or a metered dose inhaler.

11. A method according to claim 7 wherein the compound has a duration of action of 24 hours or more.
